(19) United States
(12) Patent Application Publication
(10) Pub. No.: US 2006/0148688 A1
(43) Pub. Date: Jul. 6, 2006

(54) Use of na, k-atpase a-and b-subunits in bladder cancer detection and drug screening

(76) Inventors: Ayyappan K. Rajasekaran, Los Angles, CA (US); Sigrid A. Rajasekaran, Los Angeles, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/509,784

(22) PCT Filed: Apr. 8, 2003
(86) PCT No.: PCT/US03/10826

§ 371 Date: Aug. 22, 2005

(30) Foreign Application Priority Data
Apr. 8, 2002 (US) 60371068

(51) Int. Cl. C11D 3/37; A61K 38/00

(52) U.S. Cl. 514/12

(57) ABSTRACT

Methods are provided for determining the malignancy of cells in a bladder carcinoma sample. Expression patterns of Na,K-ATPase α and β subunits are shown to be associated with recurrence risk in these cancers. Detection of Na,K-ATPase α and β subunits expression in bladder carcinomas provides a useful diagnostic for predicting patient prognosis and probability of drug effectiveness. Na,K-ATPase α and β subunits provide a target for drug screening.

### BACKGROUND OF THE INVENTION

Bladder cancer is an important clinical problem. More than 50,000 new cases of bladder cancer were diagnosed during 1999. Although 80% of cases are diagnosed early and can be effectively treated, 11,000 patients still die due to metastasis every year. About 90% of malignant tumors arising in the bladder are of epithelial origin, the majority being transitional cell carcinomas (TCC). TCC of the bladder is the second most frequent disease of the genitourinary tract and the second most prevalent cause of death of all genitourinary tumors. Low- and moderate-grade superficial lesions often recur yet seldom invade. High-grade lesions are diagnosed as invasive tumors, with significantly more life-threatening outcomes. More than 70% of treated tumors recur, and 30% of recurrent tumors advance to invasive tumors. Improved prognostication and surveillance are critical to the management of these patients.

The Na,K-ATPase catalyses an ATP-dependent transport of three sodium ions out and two potassium ions into the cell per pump cycle, thereby generating a transmembrane sodium gradient. The activity of the Na,K-ATPase is involved in the control of cellular pH, osmotic balance, and the Na⁺-coupled transport of nutrients such as amino acids and vitamins into cells. The Na,K-ATPase consists of two non-covalently linked α- and β-subunits. The α₁-subunit (˜112 kDa) contains the catalytic and ligand binding sites of the enzyme (Shull et al. (1985) Nature 316:691-95). Four α-isoforms have been described in mammals (Shamraj et al. (1994) Proc Natl Acad Sci USA 91:12952-56; Woo et al. (1999) J Membr Biol 169:39-44; Blanco et al. (1999) Biochemistry 38:13661-69); the α₁ isoform is expressed in most of the tissue types. The β₁-subunit (˜55 kDa)¹³ is a glycosylated protein and its role in Na,K-ATPase enzyme function remains somewhat obscure. It may modulate the transport of Na⁺ and K⁺ across the membrane and facilitate the insertion of the αβ-complex into the cell membrane. Of the three isoforms described (Lingrel et al. (1994) Kidney Int Suppl 1994;44:S32-39), the β₁ isoform is expressed in most of the tissues.

In renal clear-cell carcinoma the levels of Na,K-ATPase β-subunit but not the α-subunit protein levels is highly reduced and suggested that reduced expression of β-subunit might be associated with the invasiveness of the renal clear-cell carcinoma. Moloney Sarcoma Virus-transformed MDCK (MSV-MDCK) cells have highly reduced Na,K-ATPase β-subunit levels. Earlier studies correlated the invasive phenotype of MSV-MDCK cells to reduced expression of E-cadherin. In MSV-MDCK cells ectopic expression of Na,K-ATPase β-subunit and E-cadherin was necessary to induce a well differentiated phenotype and to suppress invasiveness of these cells. These studies for the first time demonstrated that Na,K-ATPase β-subunit function might play a critical role in the suppression of invasiveness of kidney carcinoma cells.

Tissue microarrays (TMA's) provide a convenient high-throughput tissue-based tool for in situ gene dosage and protein expression studies. The TMA technique has been utilized for rapid profiling of molecular tumor markers and compares well to results obtained by standard methods. TMA's are being widely used to obtain valuable information regarding the expression pattern of molecular markers in cancers and particularly in bladder carcinomas.

Biological Sequences

Na,K-ATPase is the enzyme responsible for the transport of sodium and potassium ions in most animal cells. The alpha and beta subunits of Na,K-ATPase are both encoded by multigene families. The main catalytic subunit is the α-subunit. The sequence of the human, alpha 1 genetic sequence may be found in Genbank, accession number NM_{—}000701.2. The alpha 2 (+) polypeptide (ATP1A2) genetic sequence may be found in Genbank, accession number NM_{—}000702.1. The beta 1 subunit genetic sequence may be found in Genbank, accession number NM_{—}001677.1. The beta 2 subunit genetic sequence may be found in Genbank, accession number NM_{—}001678.1. The beta 3 subunit genetic sequence may be found in Genbank, accession number NM_{—}001679.1.

### SUMMARY OF THE INVENTION

Methods are provided for prognosis of malignancy in a bladder carcinoma sample. Na,K-ATPase α and β subunit expression is shown to be associated with recurrence risk in these cancers. Patients with a high α- and low β-subunit had a high risk for early recurrence whereas patients with a low α- and high β-subunit expression had longer recurrence free time, indicating that Na,K-ATPase α- and β-subunit expression levels are useful predictors of recurrence free time distribution of bladder cancer patients. Detection of Na,K-ATPase α and β subunits expression in bladder carcinomas provides a useful diagnostic for predicting patient prognosis and probability of drug effectiveness.

Altered Na,K-ATPase α- and β-subunit levels in bladder cancer in patients can lead to reduced Na,K-ATPase activity and activation of EGFR, which contribute to the progression of bladder cancer. Therapies may alter this pattern of expression. Na,K-ATPase α and β subunits provide a target for drug screening, and for therapeutic intervention.

### BRIEF DESCRIPTION OF THE DRAWINGS

FIG. 1: Representative TMA consisting of 0.6-mm diameter tissue spots of TCC and normal urothelial tissues (Hematoxylin and Eosin). Higher power views illustrating tissue spot arrangement and an individual TMA spot.

FIG. 2: Staining of Na,K-ATPase α- (A, D,G) and β-subunit (B,E,H) and negative control (C,F,I) in matched morphologically normal (A-C) and TCC (D-I) tissues. Note the intense staining of both α- and β-subunits in the umbrella cells (arrowheads) and the basal cells (arrows). The inserts in A and B show distinct basolateral staining of Na,K-ATPase α- and β-subunit in umbrella cells. In D and E, reduced staining intensity of α- and β-subunits in a grade 1 tumor is shown. Inserts in D and E show distinct plasma membrane staining of these subunits. In G and H, increased staining of α- and β-subunits in a grade 3 tumor is shown. Inserts show both plasma membrane and cytoplasmic staining (arrowhead) of α and β-subunits. (Original objective 40X; inserts 100X).

FIG. 3. Representative immunohistochemical staining of Na,K-ATPase α- (A, D and G) and β-subunits (B,E and H) and negative control (C, F and I) on TMA. A-C, D-F, and G-I represent matched morphologically normal, grade 1 and grade 3 TMA spots, respectively. (Original objective 10X).

FIG. 4. Mean intensity staining distributions of Na,K-ATPase α- (A) and β-subunits (B) in patients stratified by histological categories. Numbers in parentheses indicate the number of tissue spots belonging to tumor of grade (1, 2, 3), CIS (carcinoma in situ), MET (metastatic tumor), NL (morphologically normal matched urothelium), DYSP (dysplastic urothelium).

FIG. 5. Kaplan-Meier curves illustrating the recurrence-free interval for patient subgroups (any stage 0a-IV; N=36) with low Na,K-ATPase α and high Na,K-ATPase β subunit expression, or high Na,K-ATPase α and low Na,K-ATPase β subunit expression. Logrank analysis comparing these subgroups showed that the high Na,K-ATPase α and low Na,K-ATPase β subunit expression subgroup had a significantly higher recurrence rate (P=0.0005).

### DETAILED DESCRIPTION OF THE EMBODIMENTS

Na,K-ATPase α-and β-subunit levels are altered during the progression of bladder cancer. Patients with a high α- and low β-subunit have a high risk for early recurrence whereas patients with a low α- and high β-subunit expression have longer recurrence free time, indicating that Na,K-ATPase α- and β-subunit expression levels are useful predictors of recurrence free time distribution of bladder cancer patients.

Expression analysis was used to test the clinical significance of Na,K-ATPase α- and β-subunits expression in a well-characterized group of patients with bladder cancer. The Na,K-ATPase subunit expression pattern were correlated with clinicopathological parameters and patient outcome to determine their potential prognostic value. Compared to the benign cells, the mean protein expression for both Na,K-ATPase α and β subunits was overall decreased in in situ and invasive tumors, as well as in premalignant dysplasic fields. When Na,K-ATPase α and β expression levels were dichotomized into distinct groups (Na,K-ATPase α< or >=85% stained frequency and beta < or >= staining intensity of 3 out of 4 possible), they were both found to be significant predictors of recurrence risk in multivariate logistic regression analysis.

Na,K-ATPase α- and β-subunit expression are the lowest in low grade tumors. Increased expression of both subunits in higher-grade tumor suggests that the mechanisms that led to reduced Na,K-ATPase α- and β-subunit expression are either inactivated, or other factors activated in higher grade tumors. In patients with relatively low α-subunit and high β-subunit expression levels, there is a significant decrease in recurrence risk and an increase in the recurrence free time. In patients with higher expression of α-subunit and low expression of β-subunit, the recurrence free time was significantly reduced, demonstrating that increased β-subunit expression has a protective effect against the recurrence of bladder cancer. In contrast, increased α-subunit expression has an unfavorable influence resulting in the increased and earlier recurrence of this cancer. Increased β-subunit expression may reduce invasiveness of tumor cells, thus reducing the chance of tumor spread and consequently its recurrence.

Without being bound by the theory of action, during bladder cancer progression, reduced β-subunit levels during the early stages of tumor development may lead to reduced Na,K-ATPase activity, resulting in events that favor the progression of bladder cancer. The intracellular sodium homeostasis, regulated by Na,K-ATPase, is crucial for the development of tight junctions and induction of polarity in epithelial cells. Tight junctions are crucial to maintain the polarized phenotype of epithelial cells. Reduced Na,K-ATPase activity during the early stages of bladder cancer may lead to loss of tight junctions and polarity in urothelial cells. Consequently, the basolaterally localized proteins, such as epidermal growth factor receptor (EGFR) may be aberrantly expressed at the apical plasma membrane. Apical expression of EGFR should allow its association with EGF present in the urine and activation of EGF mediated signaling pathways. Alternatively, the lumenal EGF might seep through the tight.junctions and activate EGFR localized to the basolateral domain. Recent studies have shown that inhibition of Na,K-ATPase can activate EGFR in a ligand independent fashion.

Where there is a dysregulation of α-subunit and β-subunit expression, for example in tumor cells, there is less active enzyme on the cell surface, because the β-subunit is involved in membrane positioning. In addition, where there is a reduced level of the β-subunit, the α-subunit may associate with other signaling molecules and induce abnormal signaling events. Therapeutic methods according to the present invention can increase activity of the ATPase enzyme, thereby correcting the intracellular sodium homeostasis, and inhibiting inappropriate activation of EGFR. Therapeutic methods can also specifically upregulate expression of the β-subunit, in order to normalize the ration between α- and β-subunit expression.

### Diagnostic Methods

Bladder carcinomas are staged according to the malignancy of the tumor, and the potential for recurrence after treatment. Earlier stages of the disease have overall lower levels of expression of the Na,K-ATPase protein, which expression level increases as the tumor advances. In one embodiment of the invention, this alteration in expression level is used to stage a bladder cancer, where reduced expression is indicative of an earlier stage of disease.

In addition, the more metastatic and malignant carcinomas have relatively high α-subunit and low β-subunit expression levels, which allows for a prognosis of the tumor outcome. By determining Na,K-ATPase α and β expression levels, and dichotomizing (Na,K-ATPase α< or >=85% stained frequency and beta < or >= staining intensity of 3 out of 4 possible), expression patterns are a significant predictor of recurrence risk in multivariate logistic regression analysis. Methods of determining which class a patient sample falls in, allows better therapeutic decisions.

Determination of a patient sample phenotype can be performed by typing the cells for the expression of Na,K-ATPase α and/or β subunits. Detection of the presence of Na,K-ATPase α and/or β subunits is performed by protein, DNA or RNA sequence and/or hybridization analysis of a patient sample. Generally the sample will be a biopsy or other cell sample from the tumor. Where the tumor has metastasized, blood samples may be analyzed if care is taken to exclude contaminating blood cells from the sample.

In a typical assay, a bladder carcinoma sample is assayed for the presence of Na,K-ATPase α and/or β subunits specific sequences by combining the sample with a Na,K-ATPase α and/or β subunits specific binding member, and detecting directly or indirectly the presence of the complex formed between the two members. The term “specific binding member” as used herein refers to a member of a specific binding pair, i.e. two molecules where one of the molecules through chemical or physical means specifically binds to the other molecule. In this particular case one of the molecules is Na,K-ATPase α and/or β subunits, where Na,K-ATPase α and/or β subunits is any protein substantially similar to the amino acid sequence of the human polypeptide sequences of this family, as described above, or a epitope containing fragment thereof. The complementary members of a specific binding pair are sometimes referred to as a ligand and receptor.

In the present specification and claims, the term “polypeptide fragments”, or variants thereof, denotes both short peptides with a length of at least two amino acid residues and at most 10 amino acid residues, oligopeptides with a length of at least 11 amino acid residues, 20 amino acid residues, 50 amino acid residues, and up to about 100 amino acid residues; and longer peptides of greater than 100 amino acid residues up to the complete length of the native polypeptide.

Polypeptides detected by the present methods include naturally occurring alpha and beta subunits, as well as variants that are encoded by DNA sequences that are substantially homologous to one or more of the DNA sequences specifically recited herein, for example variants having at least 80%, 85%, 90%, 91%, 92%, 93%, 94%, 95%, 96%, 97%, 98%, 99%, or 99.5% sequence identity. The specific binding pairs may include analogs, derivatives and fragments of the original specific binding member. For example, an antibody directed to a protein antigen may also recognize peptide fragments, chemically synthesized peptidomimetics, labeled protein, derivatized protein, etc. so long as an epitope is present.

Immunological specific binding pairs include antigens and antigen specific antibodies or T cell antigen receptors. Recombinant DNA methods or peptide synthesis may be used to produce chimeric, truncated, or single chain analogs of either member of the binding pair, where chimeric proteins may provide mixture(s) or fragment(s) thereof, or a mixture of an antibody and other specific binding members. Antibodies and T cell receptors may be monoclonal or polyclonal, and may be produced by transgenic animals, immunized animals, immortalized human or animal B-cells, cells transfected with DNA vectors encoding the antibody or T cell receptor, etc. The details of the preparation of antibodies and their suitability for use as specific binding members are well-known to those skilled in the art.

Alternatively, monoclonal or polyclonal antibodies are raised to human Na,K-ATPase α and/or β subunits. The antibodies may be produced in accordance with conventional ways, immunization of a mammalian host, e.g. mouse, rat, guinea pig, cat, dog, etc., fusion of resulting splenocytes with a fusion partner for immortalization and screening for antibodies having the desired affinity to provide monoclonal antibodies having a particular specificity. These antibodies can be used for affinity chromatography, ELISA, RIA, and the like. The antibodies may be labeled with radioisotopes, enzymes, fluorescers, chemiluminescers, or other label, which will allow for detection of complex formation between the labeled antibody and its complementary epitope.

Generally the amount of bound Na,K-ATPase α and/or β subunits detected will be compared to negative control samples from normal tissue or from known bladder cancers having a defined grade. The presence of decreased levels of Na,K-ATPase β subunit specific binding relative to expression of the alpha subunit is indicative of a more malignant tumor phenotype.

Determination of whether a patient falls into a high risk category may be performed in a number of different ways. In one embodiment, the statistical methods as set forth in the Examples are used. In another embodiment, a cohort is arranged with expression arbitrarily displayed over, for example, two logs of intensity. Cells in the lowest quadrant for expression of the beta subunit and the highest quadrant for alpha expression are assigned to the high risk group.

In an alternative method, the expression levels of the alpha and beta subunit are normalized to a control sample, e.g. to correct for antibody specificity, brightness of label, and the like, particularly when quantitative methods are used for the expression analysis. For example, one may determine the expression of the alpha and beta subunit in non-transformed cells, for example well-differentiated cell lines, which are suitable as a normal control. The ratio of α/β in such cells is generally less than 1, and a normalized ratio of less than about 1 in a cancer cell indicates a more protective phenotype. An α:β ratio of from about 1-2 indicates a lower risk, a ratio of from about 3-4 indicates a moderate risk and a ratio of about 4 or greater indicates a high risk.

Nucleic acid sequences for detection may be complementary to a Na,K-ATPase α and/or β subunit sequence. Nucleic acids complementary to Na,K-ATPase α and/or β subunits may be cDNA, mRNA or genomic DNA, or a fragment thereof. Fragments may be obtained of the DNA sequence by chemically synthesizing oligonucleotides in accordance with conventional methods, by restriction enzyme digestion, by PCR amplification, etc. For the most part, DNA fragments will be of at least 25 nt, usually at least 30 nt, more usually at least about 50 nt. Such small DNA fragments are useful as primers for PCR, hybridization screening, etc.

For use in amplification reactions, such as PCR, a pair of primers will be used. The exact composition of the primer sequences is not critical to the invention, but for most applications the primers will hybridize to the subject sequence under stringent conditions, as known in the art. It is preferable to choose a pair of primers that will generate an amplification product of at least about 50 nt, preferably at least about 100 nt. Algorithms for the selection of primer sequences are generally known, and are available in commercial software packages. Amplification primers hybridize to complementary strands of DNA, and will prime towards each other.

For hybridization probes, it may be desirable to use nucleic acid analogs, in order to improve the stability and binding affinity. The term “nucleic acid” shall be understood to encompass such analogs. A number of modifications have been described that alter the chemistry of the phosphodiester backbone, sugars or heterocyclic bases. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′S-phosphorothioate, 3′-S-5′-O-phosphorothioate, 3′-CH2-5′-O-phosphonate and 3′-NH-5′-O-phosphoroamidate. Peptide nucleic acids replace the entire phosphodiester backbone with a peptide linkage. Sugar modifications are also used to enhance stability and affinity. The a-anomer of deoxyribose may be used, where the base is inverted with respect to the natural b-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

A number of methods are available for analyzing nucleic acids for the presence or absence of a specific sequence. For analysis based on nucleic acids, mRNA or nucleic acids derived therefrom are analyzed for the presence of Na,K-ATPase α and/or β subunit specific sequences. mRNA in a sample may be used directly, or may be reverse transcribed to generate a cDNA strand. The cDNA may be amplified by conventional techniques, such as the polymerase chain reaction (PCR), to provide sufficient amounts for analysis. The use of the polymerase chain reaction is described in Saiki, et al. (1985) Science 239:487, and a review of current techniques may be found in Sambrook, et al. Molecular Cloning: A Laboratory Manual, CSH Press 1989, pp. 14.2-14.33. Amplification may also be used to determine whether a specific sequence is present, by using a primer that will specifically bind to the desired sequence, where the presence of an amplification product is indicative that a specific binding complex was formed. Alternatively, the mRNA sample is fractionated by electrophoresis, e.g. capillary or gel electrophoresis, transferred to a suitable support, e.g. nitrocellulose and then probed with a fragment of the Na,K-ATPase α and/or β subunit sequence. Other techniques may also find use, including oligonucleotide ligation assays, binding to solid state arrays, etc. Detection of mRNA having the subject sequence is indicative of Na,K-ATPase α and/or β subunit gene expression in the sample.

A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine(ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid, e.g. mRNA or amplification product, is analyzed by one of a number of methods known in the art. Hybridization with a Na,K-ATPase α and/or β subunits specific sequence may be used to determine its presence, by northern blots, dot blots, etc. The hybridization pattern of a control and variant sequence to an array of oligonucleotide probes immobilized on a solid support, as described in U.S. Pat. No. 5,445,934, or in WO95135505, may also be used as a means of detection. For examples of arrays, see Hacia et al. (1996) Nature Genetics 14:441-447; Lockhart et al. (1996) Nature Biotechnol. 14:1675-1680; and De Risi et al. (1996) Nature Genetics 14:457-460.

Screening may also be based on the functional or antigenic characteristics of the protein, e.g. immunoassays, etc. A sample is taken from a patient with bladder carcinoma. Samples, as used herein, include biological fluids such as blood; organ or tissue culture derived fluids; etc. Biopsy samples or other sources of carcinoma cells are of particular interest, e.g. tumor biopsy, etc. Also included in the term are derivatives and fractions of such cells and fluids. The number of cells in a sample will generally be at least about 10³, usually at least 10⁴, and may be about 10⁵ or more. The cells may be dissociated, in the case of solid tissues, or tissue sections may be analyzed. Alternatively a lysate of the cells may be prepared.

Detection may utilize staining of cells or histological sections, performed in accordance with conventional methods. The antibodies or other specific binding members of interest are added to the cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Final detection uses a substrate that undergoes a color change in the presence of the peroxidase. The absence or presence of antibody binding may be determined by various methods, including flow cytometry of dissociated cells, microscopy, radiography, scintillation counting, etc.

An alternative method for diagnosis depends on the in vitro detection of binding between antibodies and Na,K-ATPase α and/or β subunits in a lysate. Measuring the concentration of Na,K-ATPase α and/or β subunits binding in a sample or fraction thereof may be accomplished by a variety of specific assays. A conventional sandwich type assay may be used. For example, a sandwich assay may first attach Na,K-ATPase α and/or β subunits specific antibodies to an insoluble surface or support. The particular manner of binding is not crucial so long as it is compatible with the reagents and overall methods of the invention. They may be bound to the plates covalently or non-covalently, preferably non-covalently.

The insoluble supports may be any compositions to which polypeptides can be bound, which is readily separated from soluble material, and which is otherwise compatible with the overall method. The surface of such supports may be solid or porous and of any convenient shape. Examples of suitable insoluble supports to which the receptor is bound include beads, e.g. magnetic beads, membranes and microtiter plates. These are typically made of glass, plastic (e.g. polystyrene), polysaccharides, nylon or nitrocellulose. Microtiter plates are especially convenient because a large number of assays can be carried out simultaneously, using small amounts of reagents and samples.

Patient sample lysates are then added to separately assayable supports (for example, separate wells of a microtiter plate) containing antibodies. Preferably, a series of standards, containing known concentrations of Na,K-ATPase α and/or β subunits is assayed in parallel with the samples or aliquots thereof to serve as controls. Preferably, each sample and standard will be added to multiple wells so that mean values can be obtained for each. The incubation time should be sufficient for binding, generally, from about 0.1 to 3 hr is sufficient. After incubation, the insoluble support is generally washed of non-bound components. Generally, a dilute non-ionic detergent medium at an appropriate pH, generally 7-8, is used as a wash medium. From one to six washes may be employed, with sufficient volume to thoroughly wash non-specifically bound proteins present in the sample.

After washing, a solution containing a second antibody is applied. The antibody will bind Na,K-ATPase α and/or β subunits with sufficient specificity such that it can be distinguished from other components present. The second antibodies may be labeled to facilitate direct, or indirect quantification of binding . Examples of labels that permit direct measurement of second receptor binding include radiolabels, such as ³H or ¹²⁵I, fluorescers, dyes, beads, chemilumninescers, colloidal particles, and the like. Examples of labels that permit indirect measurement of binding include enzymes where the substrate may provide for a colored or fluorescent product. In a preferred embodiment, the antibodies are labeled with a covalently bound enzyme capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art. The incubation time should be sufficient for the labeled ligand to bind available molecules. Generally, from about 0.1 to 3 hr is sufficient, usually 1 hr sufficing.

After the second binding step, the insoluble support is again washed free of non-specifically bound material, leaving the specific complex formed between Na,K-ATPase a and b subunits and the specific binding member. The signal produced by the bound conjugate is detected by conventional means. Where an enzyme conjugate is used, an appropriate enzyme substrate is provided so a detectable product is formed.

Other immunoassays are known in the art and may find use as diagnostics. Ouchterlony plates provide a simple determination of antibody binding. Western blots may be performed on protein gels or protein spots on filters, using a detection system specific for Na,K-ATPase α and β subunits as desired, conveniently using a labeling method as described for the sandwich assay.

### Drug Screening Methods

Drug screening identifies agents that mimic or upregulate the function of Na,K-ATPase. Of particular interest are screening assays for agents that have a low toxicity for human cells. A wide variety of assays may be used for this purpose, including labeled in vitro protein-protein binding assays, electrophoretic mobility shift assays, immunoassays for protein binding, and the like. The purified protein may also be used for determination of three-dimensional crystal structure, which can be used for modeling intermolecular interactions.

The term “agent” as used herein describes any molecule, e.g. protein or pharmaceutical, with the capability of altering or mimicking the physiological function of a target protein. Generally a plurality of assay mixtures are run in parallel with different agent concentrations to obtain a differential response to the various concentrations. Typically, one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

The compounds having the desired pharmacological activity may be administered in a physiologically acceptable carrier to a host for treatment of cancer, etc., or to otherwise enhance Na,K-ATPase function. The agents may be administered in a variety of ways, orally, topically, parenterally e.g. subcutaneously, intraperitoneally, by viral infection, intravascularly, etc. Topical treatments are of particular interest. Depending upon the manner of introduction, the compounds may be formulated in a variety of ways. The concentration of therapeutically active compound in the formulation may vary from about 0.1-100 wt. %.

The agents of the present invention can be formulated according to known methods to prepare pharmaceutically useful compositions, whereby these materials, or their functional derivatives, are combined in admixture with a pharmaceutically acceptable carrier vehicle. Suitable vehicles and their formulation, inclusive of other human proteins, e.g., human serum albumin, are described, for example, in Remington's Pharmaceutical Sciences (16th ed., Osol, A., Ed., Mack, Easton, Pa. (1980)). In order to form a pharmaceutically acceptable composition suitable for effective administration, such compositions will contain an effective amount of one or more of the agents of the present invention, together with a suitable amount of carrier vehicle.

### Therapeutic/Prophylactic Treatment Methods

Na,K-ATPase α and/or β subunit coding sequences can be used to decrease the growth and/or invasiveness of bladder carcinoma cells. Embodiments of interest include the upregulation of expression in cancers in early stages, and upregulation of the β-subunit, or down-regulation of the α-subunit in more advanced stages. Expression of Na,K-ATPase α and/or β subunits is up-regulated through administration of agents that induce its expression, or through the introduction of Na,K-ATPase α and/or β subunits expression vectors into malignant tumors.

Methods can be designed to selectively deliver nucleic acids to bladder carcinoma cells. One technique for achieving selective expression in bladder carcinoma cells is to operably link the coding sequence to a promoter that is primarily active in bladder cells. Examples of such promoters include, but are not limited to, E2F promoter (Steinhoff et al. (2002) Int J Oncol 21(5):1033-40; and human uroplakin II promoter (Zhang et al. (2002) Cancer Res. 62(13):3743-50). Alternatively, or in addition, the nucleic acid can be administered with an agent that targets the nucleic acid to bladder cells. For instance, the nucleic acid can be administered with an antibody that specifically binds to a cell-surface antigen on the bladder cells or a ligand for a receptor on bladder cells.

When liposomes are utilized, substrates that bind to a cell-surface membrane protein associated with endocytosis can be attached to the liposome to target the liposome to bladder cells and to facilitate uptake. Examples of proteins that can be attached include capsid proteins or fragments thereof that bind to bladder cells, antibodies that specifically bind to cell-surface proteins on bladder cells that undergo internalization in cycling and proteins that target intracellular localizations within bladder cells (see, e.g., Wu et al. (1987) J. Biol. Chem. 262:4429-4432; and Wagner, et al. (1990) Proc. Natl. Acad. Sci. USA 87:3410-3414). Gene marking and gene therapy protocols are reviewed by Anderson et al. (1992) Science 256:808-813. Various other delivery options can also be utilized. For instance, a nucleic acid containing a sequence of interest can be injected directly into the tumor.

As used herein, the term “genetic agent” refers to polynucleotides and analogs thereof, which agents are tested in the screening assays of the invention by addition of the genetic agent to a cell. The introduction of the genetic agent results in an alteration of the total genetic composition of the cell. As employed herein, the genetic agent results in the expression of a protein and is being evaluated as to its effect on one or more target pathways. The genetic agents such as DNA result in an experimentally introduced change in the genome of a cell, generally through the integration of the sequence into a chromosome. Genetic changes can also be transient, where the exogenous sequence is not integrated but is maintained as an episomal agent. RNA viruses may be employed that comprise the gene of interest and are reverse transcribed and inserted into the genome of the host cell. Genetic agents (polypeptides or polynucleotides) can also be synthesized in vitro and delivered to cells by conjugation to a moiety (e.g antennapedia 16-amino acid “Penetratin-1 peptide, available from Qbiogene) that promotes transfer of the agent into a cell of interest. The effect of a genetic agent is to increase expression of a particular gene product in the cell with the potential for the increase and/ or decrease of other products in the cell.

Introduction of an expression vector encoding a polypeptide can be used to express the encoded product in cells to over-express the product, in the presence or absence of expression of the product. Various promoters can be used that are constitutive or subject to external regulation, where in the latter situation, one can turn on or off the transcription of a gene. These coding sequences may include full-length cDNA or genomic clones, fragments derived therefrom, or chimeras that combine a naturally occurring sequence with functional or structural domains of other coding sequences. Alternatively, the introduced sequence may encode a dominant or constitutively active mutations of native sequences; altered regulatory sequences, etc.

Methods that are well known to those skilled in the art can be used to construct expression vectors containing coding sequences and appropriate transcriptional and translational control signals for increased expression of an exogenous gene introduced Into a cell. These methods include, for example, in vitro recombinant DNA techniques, synthetic techniques, and in vivo genetic recombination. Alternatively, RNA capable of encoding gene product sequences may be chemically synthesized using, for example, synthesizers. See, for example, the techniques described in “Oligonucleotide Synthesis”, 1984, Gait, M. J. ed., IRL Press, Oxford.

A variety of host-expression vector systems may be utilized to express a genetic coding sequence. Expression constructs may contain promoters derived from the genome of mammalian cells, e.g., metallothionein promoter, elongation factor promoter, actin promoter, etc., from mammalian viruses, e.g., the adenovirus late promoter; the vaccinia virus 7.5K promoter, SV40 late promoter, cytomegalovirus, etc.

A number of viral-based expression systems may be utilized, e.g. retrovirus, lentivirus, adenovirus, herpesvirus, and the like. In cases where an adenovirus is used as an expression vector, the coding sequence of interest may be ligated to an adenovirus transcription/translation control complex, e.g., the late promoter and tripartite leader sequence. This gene may then be inserted in the adenovirus genome by in vitro or in vivo recombination. Insertion in a non-essential region of the viral genome (e.g., region E1 or E3) will result in a recombinant virus that is viable and capable of expressing the gene product in infected hosts (see Logan & Shenk, 1984, Proc. Natl. Acad. Sci. USA 81:3655-3659). Specific initiation signals may also be required for efficient translation of inserted gene product coding sequences. These signals include the ATG initiation codon and adjacent sequences. Standard systems for generating adenoviral vectors for expression on inserted sequences are available from commercial sources, for example the Adeno-X™ expression system from Clontech (Clontechniques (January 2000) p. 10-12).

In cases where an entire gene, including its own initiation codon and adjacent sequences, is inserted into the appropriate expression vector, no additional translational control signals may be needed. However, in cases where only the gene coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:516-544).

In a preferred embodiment, methods are used that achieve a high efficiency of transfection, and therefore circumvent the need for using selectable markers. These may include adenovirus infection (see, for example Wrighton, 1996, J. Exp. Med. 183: 1013; Soares, J. Immunol., 1998, 161: 4572; Spiecker, 2000, J. Immunol 164: 3316; and Weber, 1999, Blood 93: 3685); and lentivirus infection (for example, International Patent Application WO000600; or WO9851810). Adenovirus-mediated gene transduction of endothelial cells has been reported with 100% efficiency. Retroviral vectors also can have a high efficiency of infection with endothelial cells; Inaba et al (1998, J Surg Res 78:31) report 40-77% efficiency. Other vectors of interest include lentiviral vectors, for examples, see Barry et al. (2000) Hum Gene Ther 11(2):323-32; and Wang et al. (2000) Gene Ther 7(3):196-200.

Antisense molecules can be used to down-regulate expression in cells, e.g. to downregulate expression of the α-subunit. The antisense reagent may be antisense oligonucleotides (ODN), particularly synthetic ODN having chemical modifications from native nucleic acids, or nucleic acid constructs that express such antisense molecules as RNA. The antisense sequence is complementary to the mRNA of the targeted gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. It has been found that short oligonucleotides, of from 7 to 8 bases in length, can be strong and selective inhibitors of gene expression (see Wagner et al. (1996) Nature Biotechnology 14:840-844).

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in vitro or in an animal model. A combination of sequences may also be used, where several regions of the mRNA sequence are selected for antisense complementation.

Antisense oligonucleotides may be chemically synthesized by methods known in the art (see Wagner et al. (1993) supra. and Milligan et al., supra.) Preferred oligonucleotides are chemically modified from the native phosphodiester structure, in order to increase their intracellular stability and binding affinity. A number of such modifications have been described in the literature, which alter the chemistry of the backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-5′-phosphorothioate, 3′-CH2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The alpha.-anomer of deoxyribose may be used, where the base is inverted with respect to the natural .beta.-anomer. The 2′-OH of the ribose sugar may be altered to form 2′-O-methyl or 2′-0-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### EXPERIMENTAL

### EXAMPLE 1

### Analysis of the Na,K-ATPase α- and β-subunit Expression Profiles of the Bladder Cancer Using Tissue Microarrays

Na,K-ATPase α- and β-subunit protein expression patterns were analyzed using immunohistochemistry on urothelial cancer tissue microarrays (TMA) of 146 patients diagnosed with urothelial carcinoma. For each subunit, the maximum staining intensity and the percentage of positive cells staining at the maximal intensity were analyzed.

Compared to the benign fields, the mean protein expression for both Na,K-ATPase α- and β-subunit was overall decreased in in situ and invasive tumors, as well as in tumor-adjacent dysplastic fields. When Na,K-ATPase α- and β-subunit expression levels were dichotomized, both markers were found to be significant predictors of recurrence risk using multivariate logistic regression (P value=0.013, odds ratio=5.38 95% CI=[1.420, 20.368] for ATPase α, and P value=0.044, odds ratio=0.33 95% CI=[0.111, 0.972] for ATPase β) when examined together as the only covariates. In addition, both markers were found to be significant predictors of early recurrence risk in Cox multivariate analysis (P=0.0062, odds ratio=2.6 95% CI=[1.31, 5.02] and P=0.013, odds ratio=0.43 95% CI=[0.223, 0.840] for Na,K-ATPase α- and β subunits, respectively). Patients with both a high α- and low β-subunit expression had a high risk for early recurrence whereas patients with a low (β- and high β-subunit expression had a significantly longer median recurrence free time (17 months and 125 months, respectively).

These results demonstrate that Na,K-ATPase α- and β-subunit expression levels may be useful predictors of clinical outcomes such as recurrence free time of bladder cancer patients.

In the present study, we performed immunohistochemical analyses on urothelial tissue microarrays (TMA's) to test the clinical significance of Na,K-ATPase α- and β-subunit expression in a histopathologically well-characterized group of patients with TCC representing a wide spectrum of tumor grades (predominantly Grade 2 and above) and disease stages (I-IV). The Na,K-ATPase subunits expression patterns were correlated with clinicopathological parameters and patient outcome to determine their potential prognostic value.

Materials and Methods

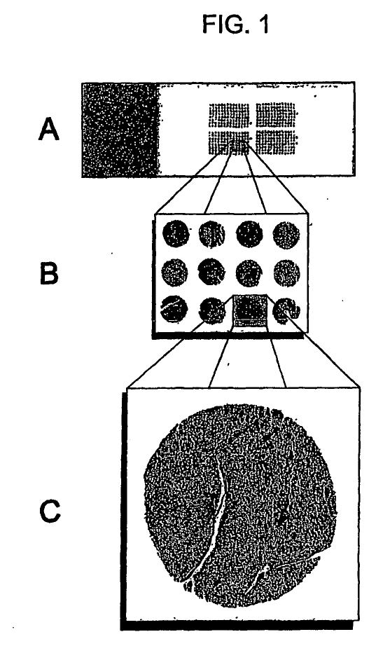

Urothelial Cancer TMA Construction. Formalin fixed, paraffin-embedded tissue samples derived from urothelial cancers between 1985 and 1995 were randomly chosen from the archives of the Department of Pathology at the University of California, Los Angeles (UCLA) Medical Center (USA) and utilized under IRB approval. The original Hematoxylin & Eosin (H&E) stained slides were reviewed by a UCLA pathologist, uniformly utilizing the 1997 TNM classification (5th ed. AJCC/UICC standards). TMA blocks were constructed following the technique described by Kononen et al (1998) Nat Med 4:844-47) using 0.6 mm diameter tissue cores arrayed into standard sized histological paraffin blocks to a density of ˜454 cores per block. Where available, at least 3 tumor and one matching morphologically normal appearing transitional epithelium sample were targeted for each case. Where available, separate 3-core sets representing dysplasia, carcinoma in situ (CIS), distinct tumor grades, or metastases were also sampled from the cases. These same arrays have been used to evaluate other biomarkers including p53, Ki67, E-cadherin and Gelsolin, and detailed information about the construction of these arrays have been previously described (Rao et al. (2002) Cancer 95(6):1247-57). An example of one of the urothelial TMA's used in this study is shown in FIG. 1.

Patients and Histopathology. Three urothelial arrays encompassing a total of 1363 tissue spots from 232 individual tumors from 167 patients with urothelial cancer were utilized. Among 167 patients, 123 patients had only one tumor, and 44 patients had multiple metachronous tumor samples (range 2-6 per patient). H&E stained array sections were histopathologically evaluated by an anatomical pathologist, in a blinded fashion to validate the diagnostic morphology of each array spot. Cases comprising only metastatic tumors and tumors showing exclusive squamous cell carcinoma or adenocarcinoma differentiation were excluded from the analysis. Post-exclusion material included 146 patients, 202 cases and 1208 tissue spots. Of the 146 urothelial tumors, 140 tumors were from bladder (2 of which showed small cell differentiation, 1 showed signet ring features, and 1 accompanied concomitant renal pelvis urothelial carcinoma), 3 were from renal pelvis alone and 3 were from ureter alone. The patient ages ranged from 33 to 94, with a mean age of 67. The male to female ratio was 3.6:1. There were 57 Tis/Ta/T1 non-invasive or superficially (lamina, propria) invasive tumors, and the remaining 89 were deeply invasive, including 44 T2, 35 T3 and 10 T4 tumors. There were 8 in situ, 6 Grade 1, 40 Grade 2 and 92 Grade 3 tumors.

Clinical and Pathology Database. Detailed retrospective demographic, pathology and clinical history information, including treatment and follow-up data for at least five years, was incorporated into a correlative database linked to the tissue specimens in an randomized fashion. Original data sources included surgical pathology reports from the UCLA Department of Pathology as well as tumor registry data obtained from the UCLA Cancer Program of the Jonsson Comprehensive Cancer Center.

Immunohistochemistry on TMA Sections. Mouse monoclonal antibodies (mAbs) raised against Na,K-ATPase α- (M7-PB-E9) and β-subunit (M17-P5-F11) recognize epitopes that are common in human, sheep, and dog and have been characterized and described previously (Rajasekaran et al. (1999) J Urol 162:574-80). A standard 2-step indirect avidin-biotin complex (ABC) method was used for immunohistochemical studies (Vector Laboratories, Burlingame, Calif.). 4 μm-thick tissue array sections were cut immediately prior to staining and were transferred using an adhesive slide system to maintain array integrity (Instrumedics, NJ) (FIG. 1A-C). They were first heated to 60° C. for 15 minutes, followed by deparaffinization in xylene. The sections were then rehydrated in graded alcohols and endogenous peroxidase quenched with 10% hydrogen peroxide in PBS at room temperature for 20 minutes. After washing, the sections were then placed in 95° C. solution of 0.01 M sodium citrate buffer for antigen retrieval. Protein blocking was accomplished through application of 1% normal horse serum, 5% BSA for 30 minutes. Primary mouse anti-Na,K-ATPase α- or β-subunit monoclonal IgG, antibodies were applied at a 1:200 dilution for 60 min at 37° C. After washing, biotinylated horse anti-mouse IgG was applied for 30 min at room temperature. The ABC complex was applied for 25 min and diaminobenzidine (DAB) was used as the chromogen. The sections were counterstained with Harris' Hematoxylin, followed by dehydration and mounting. Complementary slides processed in the same manner minus primary antibody application served as negative controls. Whole tissue sections of normal urothelial tissues served as positive tissue controls.

Histomorphologic Analysis and Scoring Criteria. The sections were analyzed with an Olympus BX-40 brightfield microscope (Olympus, Japan). Semi-quantitative assessment of antibody staining was performed blinded to clinical-pathologic variables. Scoring procedures were performed by a trained pathologist. Metrics include both maximal membrane staining intensity (graded on a 0-4 scale: 0=negative; 1=weak staining; 2=weak but distinct staining; 3=moderate staining; 4=strong staining) and the frequency of staining (proportion of the analyzed cells staining positively with the maximal intensity, 1-100%). Spots were considered informative if they were either 1) missing, 2) present but lacking target tissue or 3) damaged rendering them unreadable. If the patient had at least one evaluable tumor spot representing the patients' cancer grade, it was included in the analysis.

Statistical Analysis. The expression intensity and frequency distributions of the Na,K-ATPase α- and β-subunits were examined first by considering all informative tissue spots (662 spots for α and 678 spots for β) of 202 tumors from 146 patients. The Kruskal-Wallis test, which is a non-parametric version of one-way ANOVA for multi-group comparison, was used to compare the differences of Na,K-ATPase subunits expression between histological categories. Association between the risk of tumor recurrence and Na,K-ATPase subunit expression was analyzed using multivariate logistic regression. Recurrence was defined as returning tumor growth post-excision, seen on re-excision material or seen cystoscopically. Tumor spots in which spot grades did not match the overall case grades, CIS tumors, tumor spots which were not informative-for both Na,K-ATPase subunits, tumors of patients with no disease free interval, and with incomplete follow-up data were excluded from the outcome analyses. The resulting dataset encompassed primary tumors from 72 patients in our data set. The median age of diagnosis of this group was 67.4 years, range=[42, 82], median=69.5 with a male:female ratio of 4.5:1. 29 patients had recurrences, and 43 had no recurrence. The median follow-up time (time to first recurrence in the recurring group or total follow-up time in the non-recurring group) was 43.5 months, range [2,152]. This group contained 4 (5%) grade 1, 20 (28%) grade 2 and 48 (67%) grade 3 patients. Pathology T-stage breakdown of this group includes 13 Ta, 11 T1, 24 T2, 19 T3 and 5 T4. Pathology regional lymph node status include 26 NX, 41 NO, 4 N1, 1N2 and 0N3. 35% of the patients were group stage 1, 29% were 11, 22% III and 14% IV. No synchronous nor metachronous tumors were included in the outcome analyses. Na,K-ATPase expression intensities and frequencies from multiple spots representing the same tumor were pooled forming the mean value. Pearson correlation coefficients were used to measure correlations between the α- and β-subunit staining intensity and frequency.

The staining intensity and frequency were highly correlated for Na,K-ATPase α- but less so for β- (Pearson's correlation coefficient=0.71, P<0.0001 and 0.30, P=0.01, respectively). Therefore, in order to prevent multicolinearity problems among the subunit predictors, either intensity or frequency alone was used in further analyses. For outcome analysis, optimal correlations using the frequency metric for α- and intensity metric for β- was used. Survival analysis involving recurrence free time was conducted with recurrence time defined as the period (in months) from the date of first diagnosis to the first recurrence date or the censored date, which is the last date of negative clinical follow-up, or the date of their cystectomy. Kaplan-Meier curves were used to estimate the recurrence free time distribution. The logrank and Wilcoxon tests were used to test whether the recurrence free time distributions differed. To assess which covariates affect recurrence free time, we applied multivariate survival analysis using the Cox proportional hazards model. The proportional hazard assumption was tested using scaled Schoenfeld residuals. For each covariate, the relative hazard rate and the associated β-value were examined. For all analyses, a β-value of less than 0.05 was accepted as significant. Survival tree analysis was carried out with the rpart library in R. The analyses were carried out with the software packages R and SAS.

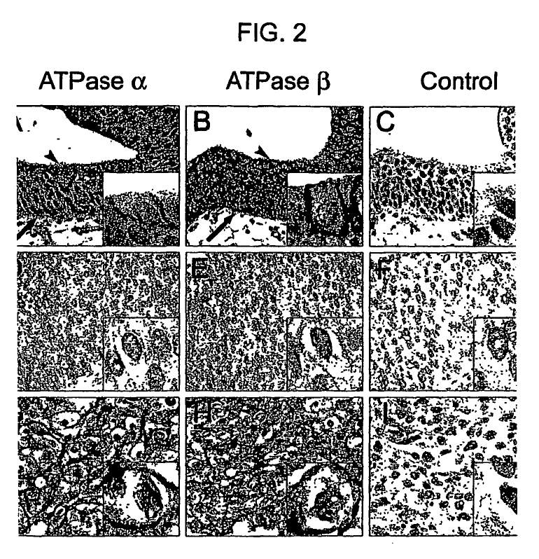

Na,K-ATPase α- and β-subunit protein expression in normal and cancer bladder tissues. Although Na,K-ATPase α- and β-subunit isoform expression pattern has been described for various tissue types, Na,K-ATPase isoforms expressed in human urinary bladder has not been reported. We found that in human urinary bladder transitional epithelium, Na,K-ATPase α₁ and β₁ isoforms are both expressed (FIG. 2). In most of the tissues, Na,K-ATPase α- and β-subunits were localized to the basolateral plasma membrane of polarized epithelial cells. In the tissue sections of morphologically normal urothelium, antibodies against Na,K-ATPase α- (FIG. 2A) and β-subunit (FIG. 2B) stained distinctly the basolateral plasma membrane of umbrella cells (see inserts in FIGS. 2A and B). In both basal cell and the intermediate transitional cell layers, Na,K-ATPase α- and β-subunit staining was uniformly distributed on the plasma membrane. The intensity of staining of the umbrella cells (arrowhead) and the basal cell layers (arrows) was more compared to the intermediate transitional cell layers. In tumor tissue sections, Na,K-ATPase α- and β-subunits showed more varied staining intensities. Examples of low and high intensity staining of both subunits are shown in representative low- and high-grade transitional cell carcinomas (FIGS. 2D and E, and 2G and H, respectively). Both the α- and β-subunits distinctly stained the plasma membrane yet some cytoplasmic staining was also detected (inserts in FIGS. 2G and H).

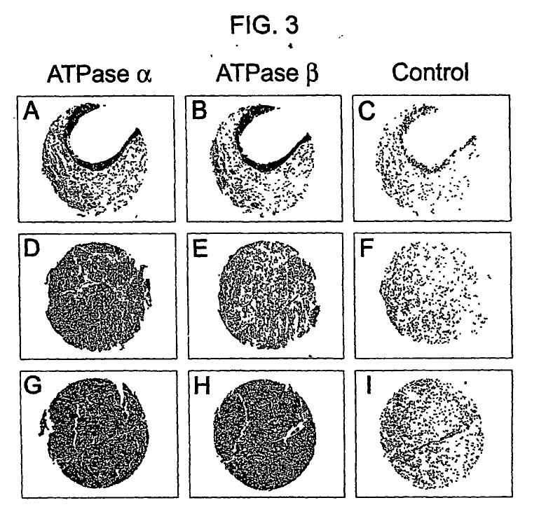

Low power views of the representative normal and tumor TMA spots showing the staining intensity of α- and β-subunits analyzed in this study are shown in FIG. 3. In normal tissue TMA spots, localization of the α- and β-subunit is clearly seen to be trans-epithelial and strongly intense (FIGS. 3 A and B). In grade 1 TMA spots (FIGS. 3D, E and F), the intensity of the α- and β-subunit was generally less compared to TMA spots representing normal tissues (FIG. 3 compare A and B with D and E). In these spots, β-subunit staining intensity was proportionally lower than that of α-subunit (FIG. 3 compare D and E). TMA spots representing grade 3 showed an intensity approaching that in normal TMA spots (FIG. 3 compare G and H with A and B).

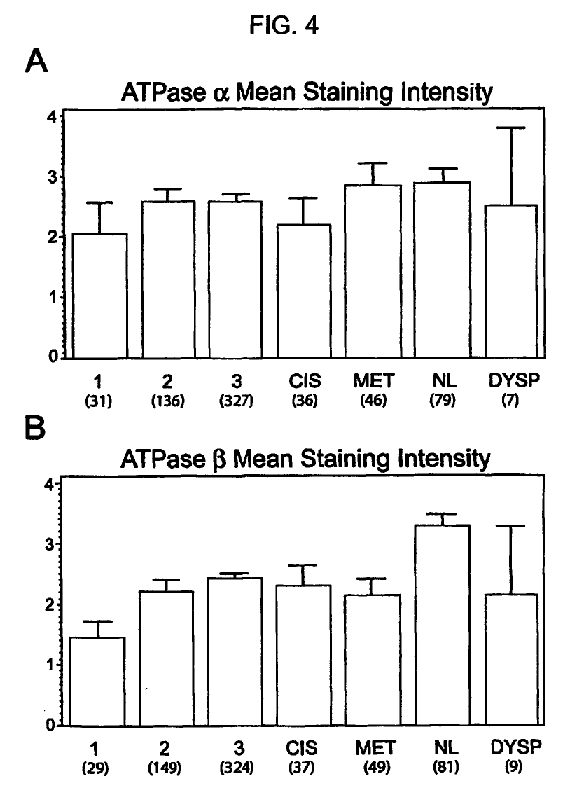

In FIG. 4, mean expression intensities with 95% confidence intervals upper limits of Na,K-ATPase α- and β-subunits from all TMA spots pooled by histological category are shown. A reduced expression of the Na,K-ATPase α- and β-subunits is seen in dysplastic and in situ lesions, as well as in tumor and their metastases, as compared to morphologically normal matched tissues from these cancer patients. Both subunits showed reduced expression in the low grade tumors (grade 1) as compared with normal tissue protein expression values (P<0.0001 by Kruskal-Wallis test for Na,K-ATPase α- and β-subunit mean expression levels). The trend of increasing expression is predominantly seen in the transition from grade 1 to 2, especially for the β-subunit, though remaining always below normal levels. There was no association between expression levels of both subunits and tumor stage.

Evaluation of the Na,K-ATPase α- and β-subunit expression in the prognosis of bladder carcinoma. The cohort of 72 patients, most of them (68) were Grade 2 and above tumors as described in Statistical Analysis, was used to analyze whether Na,K-ATPase α- and β-subunit expression was associated with tumor recurrence. Since the distribution of the staining values is highly skewed, we decided to dichotomize them. We used survival tree analysis to determine appropriate cutoff values. We found the optimal cutoff value of 85% for Na,K-ATPase α- and an intensity of 3 for Na,K-ATPase β. These dichotomized Na,K-ATPase α- or β-subunit values showed significant segregation effects on the recurrence time in univariate analyses (P=0.036, hazard ratio 1.98 95% CI=[1.05, 3.76] for α- and P=0.063, hazard ratio 0.55 95% CI=[0.292, 1.03] for β-subunit, respectively), indicating that α- and β-subunit expression in the high and low groups, respectively, is associated with a reduced time to recurrence.

In a multivariate Cox regression model utilizing the dichotomized Na,K-ATPase α- and β-subunit covariates as well as gender, age at diagnosis, and tumor grade and stage, the dichotomized Na,K-ATPase subunit expression levels were the only covariates that were significant (P=0.045, hazard ratio 2.2 95% CI=[1.02, 4.65] for α and P=0.033, hazard ratio 0.47 95% CI=[0.24, 0.94] for β). An interaction involving the α- and β-subunit expression was not significant in multivariate Cox regression indicating that their association to recurrence time is not necessarily dependent on one another.

By using the same cutoff values, we were able to divide patients into groups with significantly different recurrence free time, which we estimated with the Kaplan-Meier method. The median recurrence free time of patients with low α-subunit expression was 82 months while that of patients with high expression was 49 months (P=0,031 by logrank test). In contrast, we found that high expression of Na,K-ATPase β-subunit was protective. The median recurrence free time of patients with Na,K-ATPase β-intensity smaller than 3 was 33 months while that of patients with a high intensity was 78 months (the Wilcoxon test between the 2 groups yields P=0.026 and the logrank test P=0.061.

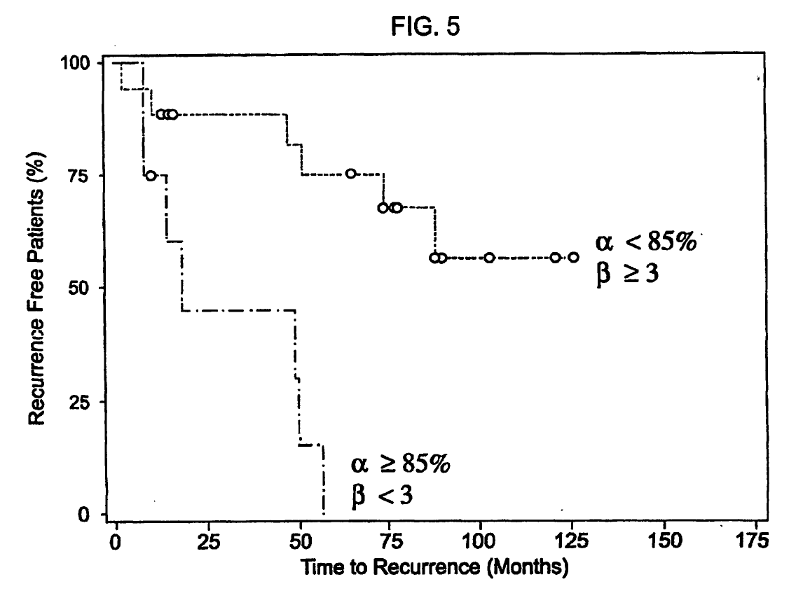

Next, we combined α- and β-subunit expression levels together to divide the patients into four groups. The first group contained patients with high α- and low β- (n=8), the second group contained patients low α- and high β- (n=18), the third group contained patients with high α- and high β (n=12), and the fourth group contained patients with low α- and low β- (n=34). Most distinctly, the median recurrence free time was 17 range=[8,57] and >126 range=[3,126] months for patients belonging to the first group and second group, respectively, with highly significant logrank test (P=0.0005), indicating that these groups form prognostically distinct patient groups (FIG. 5). The median recurrence free time for the fourth group and third group of patients was 42 months range=[3, 152] and 71 months range=[2, 87], respectively. When Na,K-ATPase expression groups one and two were stratified into low (noninvasive/group stage 0a-I) or high (invasive/group stage II-IV) stage subgroups, a similar time to recurrence curve was obtained. The high stage subgroup includes 22 patients with 17 belonging to the high β-low α and 5 patients belonging to the high α-low β groups. Low β -high α subgroup showed a recurrence free time of 48 months, range=[8,50], whereas the high β-low α-group showed a recurrence free time of >126 months, range=[3,126], (log rank test P=0.0053). While this same pattern also held for the low stage (0a-I) subgroup the number of patients was too few to generate a meaningful statistical value. These data suggest that increased α-subunit expression is associated with decreased recurrence free time while increased β-subunit expression is associated with increased recurrence free time. However, these results are based on a rather small number of patients and larger studies are necessary to further validate these findings.

Furthermore, we found that high Na,K-ATPase α-subunit and low Na,K-ATPase β-subunit measured either separately or together in the 72 patient group, lead to an increased risk of recurrence using multivariate logistic regression. Both Na,K-ATPase α- and β-subunit values were significant (P value=0.013, odds ratio=5.38 95% CI=[1.420, 20.368] for α and P value=0.044, odds ratio=0.33 95% CI=[0.111, 0.972] for β) when examined together as the only covariates. When other covariates such as gender, age, grade, and stage were added into the logistic regression model, the Na,K-ATPase α- and β-subunit expression levels lost significance (P value=0.06, odds ratio=3.98 95% CI=[0.943,16.824] for α- and P value=0.066, odds ratio=0.32 95% CI=[0.092, 1.077] for β).

Utilizing TMA technology, we have studied the protein expression patterns of Na,K-ATPase α- and β-subunits in urothelial cancer patients. Our study revealed a distinctive bi-phasic protein expression pattern of the Na,K-ATPase α- and β-subunits in urothelial cancer. Expression intensity is highest for both subunits in morphologically normal tissues. Significant decreases in expression are seen in the limited number of adjacent dysplasia and grade 1 papillary tumors, suggesting that reduced Na,K-ATPase subunit levels might have occurred at the early stage of tumor development. The lowest expression is seen in grade 1 tumors, with tumors of higher grade displaying increasing expression. This bi-phasic pattern of Na,K-ATPase subunit expression in bladder cancer suggests that Na,K-ATPase subunits levels and possibly the Na,K-ATPase activity are modulated during bladder cancer progression. We have shown earlier that in renal clear-cell carcinoma, although near normal levels of α-subunit were present, reduced levels of β-subunit in tumor tissues correlated with significantly reduced Na,K-ATPase activity.

Reduced Na,K-ATPase activity has been shown during the progression of colon cancer in animal models. During bladder cancer progression, reduced β-subunit levels during the early stages of tumor development may lead to reduced Na,K-ATPase activity, resulting in events that favor the progression of bladder cancer. The intracellular sodium homeostasis, regulated by Na,K-ATPase, is crucial for the development of tight junctions and induction of polarity in epithelial cells. Tight junctions are crucial to maintain the polarized phenotype of epithelial cells. Reduced Na,K-ATPase activity during the early stages of bladder cancer may lead to loss of tight junctions and polarity in urothelial cells. Consequently, the basolaterally localized proteins, such as epidermal growth factor receptor (EGFR) may be aberrantly expressed at the apical plasma membrane. Apical expression of EGFR should allow its association with EGF present in the urine and activation of EGF mediated signaling pathways. Alternatively, the lumenal EGF might seep through the tight junctions and activate EGFR localized to the basolateral domain. Recent studies have shown that inhibition of Na,K-ATPase can activate EGFR in a ligand independent fashion.

Altered Na,K-ATPase α- and β-subunit levels in bladder cancer may lead to reduced Na,K-ATPase activity and activation of EGFR, which could further contribute to the progression of bladder cancer.

In this study, we found that Na,K-ATPase α- and β-subunit expression are the lowest in low grade tumors. Both Na,K-ATPase α- and β-subunits are known to be regulated transcriptionally as well as translationally in a wide variety of cell types. It is possible that factors induced during the early stage of tumor development may transcriptionally or translationally reduce the expression of Na,K-ATPase subunits. Increased expression of both subunits in higher-grade tumor suggests that the mechanisms which led to reduced Na,K-ATPase α- and β-subunit expression are either inactivated probably by newly induced factors or activation of existing factors in higher grade tumors. Alternatively, reduced Na,K-ATPase activity in low grade tumors might lead to an increase in the intracellular sodium concentration. It has been shown that increased intracellular sodium can increase the transcription of both α- and β-subunits thus increasing the levels of these mRNAs and proteins in higher grade cancers.

In patients with low α-subunit and high β-subunit expression levels, we found a significant decrease in recurrence risk and an increase in the recurrence free time. On the other hand, in patients with higher expression of α-subunit and low expression of β-subunit, the recurrence free time was significantly reduced, suggesting that increased β-subunit expression has a protective effect against the recurrence of bladder cancer. In contrast, increased α-subunit expression appears to have an unfavorable influence resulting in the increased and earlier recurrence of this cancer. We have shown earlier that in E-cadherin expressing MSV-MDCK cells, expression of the β-subunit induced the formation of junctional complexes such as tight junction and desmosomes and significantly reduced the motility and invasiveness of these cells. These studies revealed that β-subunit of Na,K-ATPase might be involved in the mechanisms leading to the suppression of invasiveness of carcinoma cells. It is possible that increased β-subunit expression might reduce invasiveness of tumor cells, thus reducing the chance of tumor spread and consequently its recurrence.

Both α- and β-subunits can be regulated by independent mechanisms. Significantly different levels of α- and β-subunit in bladder carcinoma patients, as well as their statistical lack of interaction in multivariate analyses, suggest that in human urothelium, these subunits are probably differentially regulated. Identification of these mechanisms should facilitate novel therapeutic approaches to treat bladder carcinoma.

When patients are dichotomized into high and low Na,K-ATPase α- and Na,K-ATPase β-subunit expression subgroups, these markers were-more significant predictors of recurrence risk and recurrence time than either the stage or grade of the tumor, indicating that these markers could provide a significant addition as clinically useful prognosticators. These findings indicate that Na,K-ATPase α- and Na,K-ATPase β subunits may form a set of potentially useful tumor markers that may help guide therapeutic decision-making, and could serve as promising therapeutic targets.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for analysis of a bladder carcinoma, the method comprising:
detecting the presence of Na,K-ATPase α and/or β subunit in said carcinoma, wherein presence of high levels of the Na,K-ATPase cc subunit and low levels of β subunit is indicative of increased risk for malignancy.

2. The method according to claim 1, wherein said detection comprises determining the presence of a nucleic acid encoding Na,K-ATPase α and/or β subunit.

3. The method according to claim 1, wherein said detection comprises determining the presence of Na,K-ATPase α and/or β subunit polypeptide.

4. The method according to claim 1, wherein said α-subunit is human α1-subunit.

5. The method according to claim 1, wherein said β-subunit is human β1-subunit.

6. The method according to claim 1, further comprising the step of determining the ratio between expression of said α-subunit, and said β-subunit, and normalizing said ratio to expression in a control, non-transformed cell.

7. The method according to claim 6, wherein a normalized ratio of greater than about 3 is indicative of a higher risk of tumor recurrence.

8. The method according to claim 1, further comprising the step of analyzing combined Na,K-ATPase α and β subunit expression, and wherein a level of expression that is reduced compared to a control sample Is indicative of an early stage cancer.

9. A method of screening for agents that affect the malignancy of bladder carcinoma, the method comprising:
contacting a candidate biologically agent with a Na,K-ATPase α and/or β subunit or a cell expressing said Na,K-ATPase α and/or β subunit and determining the effect of said agent.

10. The method according to claim 9; wherein said biologically active agent upregulates activity of said Na,K-ATPase.

11. The method according to claim 9, wherein said biologically active agent upregulates expression of said Na,K-ATPase β subunit.

12. The method according to claim 9, wherein said biologically active agent downregulates expression of said Na,K-ATPase α subunit.

13. The method according to claim 9, wherein said biologically active agent modulates activity of said polypeptide.

14. A method of decreasing the malignancy of a bladder carcinoma, the method comprising:
enhancing the activity of Na,K-ATPase in said carcinoma.

15. The method according to claim 14, wherein said bladder cancer has an increased risk of recurrence by the method set forth in claim 7.

16. The method according to claim 15, comprising the step of increasing expression of Na,K-ATPase β-subunit.

17. The method according to claim 15, comprising the step of decreasing expression of Na,K-ATPase α-subunit.

18. The method according to claim 14, wherein said method decreases activation of the epidermal growth factor receptor in said bladder cancer.
